(19) United States
(12) Patent Application Publication
(10) Pub. No.: US 2006/0154855 A1
(43) Pub. Date: Jul. 13, 2006

(54) Methods and compositions for beta conglycinin fraction of soy protein

(75) Inventors: Michael R. Adams, Pfafftown, NC (US); Mary S. Anthony, Thousand Oaks, CA (US)

Correspondence Address: MYERS BIGEL SIBLEY & SAJOVEC, PO BOX 37428, RALEIGH, NC, 27627, US

(73) Assignee: Wake Forest University

(21) Appl. No.: 11/305,530

(22) Filed: Dec. 16, 2005

Related U.S. Application Data
(63) Continuation of application No. 11/229,750, filed on Sep. 19, 2005, now abandoned.
(60) Provisional application No. 60/611,530, filed on Sep. 20, 2004.

(51) Int. Cl. A61K 38/16

(52) U.S. Cl. 514/8

(57) ABSTRACT

The present invention provides compositions and methods useful for preventing and/or treating a cardiovascular disorder such as atherosclerosis, coronary heart disease, myocardial infarction, and stroke in a subject, comprising administering to the subject an effective amount of β-conglycinin, with or without soy isoflavones.

### STATEMENT OF PRIORITY

This application is a continuation application of U.S. application Ser. No. 11/229,750, filed Sep. 19, 2005, now abandoned, which claims the benefit, under 35 U.S.C. § 119(e), of U.S. Provisional Application Ser. No. 60/611,530, filed Sep. 20, 2004, the entire contents of each of which are incorporated by reference herein.

### STATEMENT OF GOVERNMENT SUPPORT

The present invention is supported by U.S. Government Grant No. 5RO1HL064746-04 awarded by the National Institutes of Health. The Government has certain rights in this invention.

### FIELD OF THE INVENTION

The present invention provides nutritional supplements and food compositions comprising β conglycinin and methods of their use in the treatment of various disorders, such as atherosclerosis.

### BACKGROUND OF THE INVENTION

The major soy storage proteins are contained in the 7S, or β-conglycinin, and 11S, or glycinin, fractions. The 7S fraction (MW ˜200 kDa) is a trimeric glycoprotein composed of random combinations of three subunits, α (57 to 76 kDa), α (57 to 83 kDa) and β (42 to 53 kDa), the peptide compositions of which are incompletely known. It accounts for about 30% of all soy protein. Certain subfractions of 7S are rich in peptide sequences that are preserved despite the hydrolytic process and are absorbed from the gastrointestinal (GI) tract.

The present invention demonstrates a potent anti-atherosclerotic effect of soy β-conglycinin fraction. These findings indicate that the use of products enriched in this protein fraction can reduce the incidence and/or severity of cardiac disease and other disorders Thus, the present invention provides compositions and methods directed to providing these results.

### SUMMARY OF THE INVENTION

The present invention provides a method of preventing and/or treating a cardiovascular disorder such as atherosclerosis, coronary heart disease, myocardial infarction, and/or stroke, typically in a subject in need thereof, comprising administering to the subject an effective amount of β-conglycinin, with or without soy isoflavone.

Further provided herein is a method of treating and/or preventing an inflammatory condition in a subject, comprising administering to the subject an effective amount of β conglycinin, with or without soy isoflavone.

Additionally provided are 1) methods of treating and/or preventing osteoporosis, 2) methods of treating and/or preventing obesity, 3) treating and/or preventing diabetes and 4) lowering and/or maintaining blood pressure in a subject, which may be a subject in need thereof, comprising administering to the subject an effective amount of β-conglycinin.

Also provided herein is a method of improving memory and/or cognitive function in a subject, which may be a subject in need thereof, comprising administering to the subject an effect amount of β-conglycinin.

In further embodiments, the present invention provides a method of treating and/or preventing breast and/or uterine and/or prostate cancer in a subject, which can be a subject in need thereof, comprising administering to the subject an effective amount of β-conglycinin.

In further embodiments, the present invention provides a food composition, comprising, per serving or unit, at least about 1 to at least about 1000 grams of β-conglycinin, with or without soy isoflavones.

### BRIEF DESCRIPTION OF THE DRAWINGS

FIG. 1. Aortic cholesteryl ester levels of male apoE −/− mice fed diets with different sources of protein. The molecular weight of the cholesteryl ester standard is 650. C/L, casein/lactalbumin; soy+, isoflavone-containing soy protein; W008, a precipitated peptide fraction of hydrolyzed soy protein. Values are means±SEM, n=11-24. Bars labeled with different letters differ (P<0.05).

FIG. 2. Aortic cholesterol ester levels of male LDL receptor −/− mice fed diets with different sources of protein. The molecular weight of the cholesteryl ester standard is 650. C/L, casein/lactalbumin; soy+, isoflavone-containing soy protein; W008, a precipitated peptide fraction of hydrolyzed soy protein. Values are means±SEM, n=10-24. Bars labeled with different letters differ (P<0.05).

FIG. 3. Aortic cholesteryl ester levels of female apoE −/− mice fed diets with different sources of protein. The molecular weight of the cholesteryl ester standard is 650. C/L, casein/lactalbumin; soy+, isoflavone-containing soy protein; W008, a precipitated peptide fraction of hydrolyzed soy protein. Values are means±SEM, n=11-24. Bars labeled with different letters differ (P<0.05).

FIG. 4. Aortic cholesteryl ester levels of female LDL receptor −/− mice fed diets with different sources of protein. The molecular weight of the cholesteryl ester standard is 650. C/L, casein/lactalbumin; soy+, isoflavone-containing soy protein; W008, a precipitated peptide-fraction of hydrolyzed soy protein. Values are means±SEM, n=10-23. Bars labeled with different letters differ (P<0.05).

### DETAILED DESCRIPTION OF THE INVENTION

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The present invention is based on the unexpected discovery that the β conglycinin (7S) fraction of soy protein has a potent anti-atherosclerotic effect. Thus, in one embodiment, the present invention provides a method of preventing and/or treating a cardiovascular disorder, which can be, but is not limited to, atherosclerosis, coronary heart disease, myocardial infarction, and stroke, in a subject, which can be a subject in need thereof, as well as a subject at risk of developing a cardiovascular disorder, comprising administering to the subject an effective amount of β-conglycinin.

Further provided herein is a method of treating and/or preventing an inflammatory condition in a subject, which can be a subject in need thereof, as well as a subject at risk of developing an inflammatory condition, comprising administering to the subject an effective amount of β-conglycinin. The relationship between inflammatory processes and cardiovascular disorders such as atherosclerosis has been described in the literature (see, e.g., Willerson and Ridker (2004) “Inflammation as a cardiovascular risk factor” Circulation 109(21 Suppl 1):II2-10; Shishehbor and Hazen “Inflammation and oxidative markers in atherosclerosis. Relationship to outcome” Curr. Atheroscler. Rep. 6:243-250). Thus, the studies described in, the Examples herein demonstrate that the mechanism of the effect of β-conglycinin on preventing atherosclerosis is at the level of preventing an inflammatory condition and therefore the present invention provides methods of treating and/or preventing an inflammatory condition in a subject.

The present invention additionally provides a method of treating and/or preventing osteoporosis in a subject, which can be a subject in need thereof, as well as a subject identified to be at risk of developing osteoporosis, comprising administering to the subject an effective amount of β-conglycinin.

In further embodiments, the present invention provides a method of treating and/or preventing obesity in a subject, which can be a subject in need thereof, as well as a subject at risk of becoming obese, comprising administering to the subject an effective amount of, conglycinin.

Additionally, the present invention provides a method of treating and/or preventing diabetes in a subject, which can be a subject in need thereof, as well as a subject at risk of developing diabetes, comprising administering to the subject an effective amount of β-conglycinin.

Also provided herein is a method of lowering or maintaining blood pressure in a subject, which can be a subject in need thereof, as well as a subject at risk of developing abnormal or high blood pressure, comprising administering to the subject an effective amount of β-conglycinin.

In yet further embodiments herein, the present invention provides a method of improving memory and/or cognitive function in a subject, which can be a subject in need thereof, as well as a subject at risk of developing problems with memory and/or cognitive function, comprising administering to the subject an effect amount of β-conglycinin.

It is also contemplated that the compositions of this invention can have a therapeutic effect in the treatment and/or prevention of certain types of cancer. Thus, the present invention further provides a method of treating breast and/or uterine and/or prostate cancer in a subject, which can beta subject in need thereof, as well as a subject at risk of developing breast and/or uterine and/or prostate cancer, comprising administering to the subject an effective amount of β-conglycinin.

Further provided are methods of maintaining health or cardiac health, comprising ingesting the compositions of this invention as a dietary supplement. In this embodiment, a subject in good health and/or in good cardiac health (i.e., a subject not in need of any of the treatment or prevention described herein) would ingest the food composition or dietary supplement of this invention on a regular basis (e.g., daily), for an extended period (e.g., months to years) to achieve the health benefits described herein, e.g., to maintain a status of good health and/or a status of good cardiac health.

The subject of this invention can be any animal that is susceptible to the disorders and diseases described herein (e.g., cardiovascular disorders, etc.) that can be treated and/or prevented by the administration of β conglycinin. In certain embodiments, the subject is a mammal and in particular embodiments, the subject is a human. Examples of other mammals of this invention include non-human primates, dogs, cats, horses, cows, sheep, pigs, rabbits, mice, rats, and any other domestic or commercially useful mammal. The subjects can be of any age, including infant, juvenile, adolescent and adult, with the dosage or amount of the food composition administered adjusted appropriately. Subjects described herein as being at risk of developing the various disorders or diseases that are the subjects of the methods described herein are identified by family history, genetic analysis, environmental exposure and/or the onset of early symptoms associated with the disease or disorder described herein.

In the methods of this invention, the amount of β-conglycinin administered can be a dosage amount in the range of about 0.1 gram to about 1000 grams. Thus, for example, the dose of β-conglycinin can be 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 or more grams. In some embodiments, the dose of β-conglycinin can be administered to a subject of this invention at least once, twice, three, four, five, six, seven, eight, nine, ten, or more times in a day, which can be every consecutive day for a period of days, or, alternating days or any other schedule of administration by days. In addition or alternatively, the β-conglycinin can be administered at least once, twice, three, four, five, six, seven, eight, nine, ten, or more times weekly. The duration of administration can be in days, weeks, months, or years. For example, to treat or prevent various disorders described herein, a dose of about 2, 3, 4 or 5 grams to about 100, 150 or 200 grams is administered daily for a period of three months, six months, nine months, or up to one year or longer. The subject's clinical condition is evaluated at that time to determine if further administration of β-conglycinin is indicated and if the dose and/or frequency of administration should be decreased or increased, as would be readily determined by one skilled in the art.

In some embodiments of this invention, the β-conglycinin is administered to the subject of this invention in combination with soy isoflavone. In some embodiments of this invention, the β-conglycinin is administered to the subject in the absence of any soy isoflavones or any detectable amount of soy isoflavones. The soy isoflavone can be administered to the subject before, after and/or simultaneously with the administration of the β-conglycinin in the methods of this invention. Thus, when soy isoflavone is administered to a subject in the methods of this invention, the amount of soy isoflavone can be in the range of about 0.1 milligram to about 1000 milligrams. Thus, the amount of soy isoflavone can be, for example, 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 milligrams. In some embodiments, the dose of soy isoflavone can be administered to a subject of this invention at least once, twice, three, four, five, six, seven, eight, nine, ten, or more times in a day, which can be every consecutive day for a period of days, or alternating days or any other schedule of administration by days. In addition or alternatively, the soy isoflavone can be administered at least once, twice, three, four, five, six, seven, eight, nine, ten, or more times weekly. The duration of administration can be in days, weeks, months, or years. For example, to treat or prevent various disorders described herein, a dose of about 1, 2 or 5 milligrams to about 100, 150 or 200 milligrams is administered daily for a period of three months, six months, or nine months, or up to one year or longer. The subject's clinical condition is evaluated at that time to determine if further administration of isoflavone is indicated and if the dose and/or frequency of administration should be decreased or increased, as would be readily determined by one skilled in the art.

An effective amount is an amount of β-conglycinin, soy isoflavone or a combination of β-conglycinin and soy isoflavone that is sufficient to produce a desired effect, which can be a therapeutic or beneficial effect. The effective amount will vary with the age, general condition of the subject, the severity of the condition being treated, the particular biologically active agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science And Practice of Pharmacy (20th ed. 2000)).

Also as used herein, the terms “treat,” “treating” and “treatment” include any type of mechanism, action or activity that results in a change in the medical status of a subject, including an improvement in the condition of the subject (e.g., change or improvement in one or more symptoms and/or clinical parameters), delay in the progression of the condition, prevention or delay of the onset of a disease or illness, etc.

In the methods of this invention, the β-conglycinin and/or soy isoflavone are typically administered to the subject orally, e.g., in the form of a food composition. The composition can be in the form of a powder that is added to food or beverages or the composition can be a component of a solid, liquid and/or semi-liquid food composition that is incorporated into the food composition during its manufacture.

In some embodiments, the present invention provides a food composition or dietary supplement, which can be a solid food composition in the form of a bar, cookie, wafer, loaf, cracker, cake or the like as would be readily known to one skilled in the art. The food composition of this invention can also be a liquid or semi-liquid food composition in the form of a beverage, yogurt, “smoothie,” milkshake, ice cream, sherbet, pudding, custard, gelatin, beverage concentrate or beverage mix. The food composition of this invention can also be a powder or gel composition that can be added to and/or incorporated into other food compositions and/or ingested separately as a dietary supplement. The dietary supplement can further comprise ingredients such as enzymes, a fiber source, vitamins and the like.

The main component of the food composition of this invention is β conglycinin, which can be present in the composition in an amount per unit or per serving in an amount ranging from about 1 gram to about 1000 grams. Thus, the composition can comprise, per unit or per serving at least about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, 400, 425, 450, 475, 480, 485, 490, 495 or 500, 550, 600, 650, 700, 750, 800, 850, 900, 950 or 1000 grams of β conglycinin, including any value in between the values recited herein. The β conglycinin can also be present in the composition of this invention as a percent amount of the total composition. Thus, the β conglycinin can make up at least about 5, 10, 20, 30, 40, 50, 60, 70, 80, 90 or 95% of the total weight of the composition, including any percentages in between the percentages recited herein. Additionally or alternatively, the β conglycinin can make up a percentage of a protein component of the composition of this invention. Thus, the β conglycinin can make up at least about 5, 10, 20, 30, 40, 50, 60, 70, 80, 90, 95, 98 or 100% of the protein portion of the composition of this invention, including any percentages in between the percentages recited herein. The β conglycinin can also be present in the composition in any percentage of the protein of the composition and can be defined in terms of greater than or less than. For example, the food composition of this invention can comprise an amount of β conglycinin per unit or serving that is greater than 80% or less than 40% of the protein of the composition.

Thus, it is also understood that the β conglycinin can be present in the food composition of this invention in any range that is greater than or equal to or at least one value and less than or equal to another value. As one non-limiting example, the β conglycinin can be present in the composition in an amount that is at least 10% of the composition and not greater than 30% composition, or as another non-limiting example, the β conglycinin can be present in the composition in an amount that is at least 10 grams and not greater than 50 grams of the total weight of the composition. These are non-limiting examples only and any value provided herein, or numbers in between the specifically recited values provided herein could be substituted to define the ranges described herein.

The food composition can further comprise a soy isoflavone. The food composition can also be devoid of a soy isoflavone or any detectable amount of a soy isoflavone. The food composition can also comprise a plurality of isoflavones, including, but not limited to, diadzin, genistin, glycitin, diadzein, genistein, glycitein and equol in any combination and in any ratios relative to one another. For example, in one embodiment, the food composition can comprise diadzin, genistin and glycitin in a diadzin to genistin to glycitin ratio of between 3:1:2 and 3:4.5:1. In other embodiments, for example, the food composition can comprise a ratio of diadzin to genistin to glycitin of near or approximately 2:1:1, respectively.

The food composition can thus, in some embodiments, comprise, in addition to the β conglycinin described herein, an amount of an isoflavone per unit or per serving that is at least about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, 400, 425, 450, 475, 480, 485, 490, 495 or 500, 550, 600, 650, 700, 750, 800, 850, 900, 950 or 1000 milligrams, including any value in between the values recited herein. The soy isoflavone(s) can also be present in the composition of this invention as a percent amount of the total composition. Thus, the soy isoflavone(s) can make up at least about 5,10, 20, 30, 40, 50, 60, 70, 80, 90 or 95% of the total weight of the composition. Additionally or alternatively, the soy isoflavone(s) can make up a percentage of a protein component of the composition of this invention. Thus, the soy isoflavone(s) can make up at least about 5, 10, 20, 30, 40, 50, 60, 70, 80, 90, 95, 98 or 100% of the protein portion of the composition of this invention, including any percentage in between the percentages recited herein. The soy isoflavone(s) can also be present in the composition in an amount that recites a ratio relative to the amount of β conglycinin in the composition. Thus, the soy isoflavone(s) and β conglycinin can be present in a composition of this invention in a ratio of 5000:1. 4000:1. 3000:1. 2000:1, 1500:1, 1000:1, 900:1, 800:1, 700:1, 600:1, 500:1, 400:1, 300:1, 200:1, 100:1, 50:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:50, 1:100, 1:200, 1:300, 1:400, 1:500, 1:600, 1:700, 1:800, 1:900, 1:1000, 1:1500, 1:2000, 1:3000, 1:4000, or 1:5000 soy isoflavone: β conglycinin. In some embodiments of this invention, the food composition is devoid of soy isoflavone or of a particular soy isoflavone.

It is also understood that the soy isoflavone can be-present in the food composition of this invention in any range that is greater than or equal to or at least one value and less than or equal to another value. As one non-limiting example, the soy isoflavone can be present in the composition in an amount that is at least 10% of the composition and not greater than 30% composition, or as another non-limiting example, the soy isoflavone can be present in the composition in an amount that is at least 10 milligrams and not greater than 50 milligrams of the total weight of the composition. These are non-limiting examples only and any value provided herein, or numbers in between the specifically recited values provided herein could be substituted to define the ranges described herein.

A unit or serving size of the food composition of this invention can be about 1 gram to about 1000 grams. Thus, a unit or serving size can be, for example, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, 400, 425, 450, 475, 480, 485, 490, 495 or 500, 550, 600, 650, 700, 750, 800, 850, 900, 950 or 1000 grams.

The composition of this invention can comprise, in addition to the β conglycinin, and optionally the isoflavone(s), other proteins, or the β conglycinin and optional isoflavone(s) may be the total protein source of the food composition. Additional proteins that can be included in the composition of this invention include, but are not limited to, peanut proteins, such as peanut flours and grits, milk products, non-fat dry milk solids, soy proteins, whey proteins, wheat proteins such as wheat germ, casein hydrolysate and caseinates, such as potassium, sodium and calcium caseinate. In some embodiments, the food composition is devoid of additional protein besides β conglycinin or of a particular type of additional protein.

The composition of this invention can further comprise a sweetening agent, which can be, but is not limited to dextrose, maltodextrin, tagatose, invert sugar, sucrose, maltose, lactose, glucose, galactose, fructose, sugar alcohols (e.g., sorbitol, malitol), artificial sweeteners (e.g., saccharin, sucralose, aspartame, acesulfame potassium, sodium cyclamate) and plant derived sweeteners including stevia and can be in the form of sugar granules (white, light brown, dark brown, etc.), powdered sugar, honey, maple syrup, corn syrup, rice syrup solids, molasses, confectioner's coating (e.g., comprising sugar, fat, optionally milk) and the like. The sweetening agent can be present in the composition in an amount that is at least about 1, 2, 3, 4, 5, 6, 7, 8, 9, 10, 15, 20, 25, 30, 35, 40, 45, 50, 60, 70, 80, 90 or 95% of the total weight of the composition, including any percentages between the percentages recited herein. In some embodiments, the food composition is devoid of a sweetening agent or of a detectable amount of a sweetening agent or of a particular sweetening agent.

It is also understood that the sweetening agent can be present in the food composition of this invention in any range that is greater than or equal to or at least one value and less than or equal to another value. As one non-limiting example, the sweetening agent can be present in the composition in an amount that is at least 10% of the composition and not greater than 30% composition. This is a non-limiting example only and any value provided herein, or numbers in between the specifically recited values provided herein could be substituted to define the ranges described herein.

The composition can also comprise a carbohydrate component, which may be the sweetening agent, or may be in place of the sweetening agent, or may be in addition to the sweetening agent. Examples of carbohydrates that can be included in the food composition of this invention include but are not limited to sugar, starches, polydextrose, polysaccharides, dextrin, maltodextrin, corn syrup solids, rice, oats, corn, rye, barley, wheat, hominy and combinations thereof. The carbohydrate(s) can be present in the composition in an amount that is at least about 1, 2, 3, 4, 5, 10, 15, 20, 30, 40, 50, 60, 70, 80, 90 or 95% of the total weight of the composition, and any percentages between the percentages recited herein. In some embodiments, the food composition is devoid of a carbohydrate or a detectable amount of a carbohydrate or of a particular carbohydrate.

It is also contemplated that the composition of this invention can comprise a fat, which can be, for example, a solid fat, such as butter, cocoa butter, peanut butter and other nut butters, margarine hydrogenated cottonseed, coconut, soybean, palm and/or peanut oil and/or vegetable shortening, or a liquid oil, such as vegetable oil, soybean oil, cottonseed oil, sunflower seed corn oil and/or palm oil, in any combination. The food composition can also comprise a fat in the form of an animal fat. The fat can also be in the form of nuts, such as, for example, peanuts, walnuts, pecans, cashews, almonds, pistachios, etc. The fat(s) can be present in the composition in an amount that is at least about 1, 2, 3, 4, 5, 10, 15, 20, 30, 40, 50, 60, 70, 80, 90 or 95% of the total weight of the composition or any percentage between the percentages recited herein. In some embodiments, the food composition is devoid of a fat or of a detectable amount of a fat or of a particular fat.

The food composition can further comprise dietary fiber, which can be, for example, in the form of rolled oats and brans or it can be psyllium. The dietary fiber can be present in the composition in an amount that is at least about 1, 2, 3, 4, 5, 10, 15, 20, 30, 40, 50, 60, 70, 80, 90 or 95% of the total weight of the composition, or any percentage between the percentages recited herein. In some embodiments, the food composition is devoid of dietary fiber or any detectable amount of dietary fiber or of a particular type of dietary fiber.

Additionally, the food composition of this invention can comprise vitamins and minerals, which may be coated or uncoated. Vitamins and minerals that are available commercially as premixes, together and separately, can be used in the food composition of this invention. The amount of the vitamins can be at least about 0.5, 1, 2, 3, 4, 5, 6, 7, 8, 9, or 10% of the total weight of a unit or serving of the food composition or any percentage between the percentages recited herein. The food compositions can also be devoid of added vitamins and minerals or of any detectable amount of vitamins and minerals or of particular vitamins and minerals.

Salt and other seasonings can be included in the food composition, as well as various flavorings, which can be, for example, chocolate, vanilla, fruit, nut, caramel, maple, coconut, butter, coffee, toffee, butterscotch, and/or mint flavorings. The food composition can also be devoid of added salt and seasonings or added flavorings. The food composition can be uncoated or can be coated in an icing (e.g., chocolate, vanilla) or confectioner's coating, which may contain milk products or may be lactose-free. The compositions of typical coating materials that can be used are disclosed in Matz, Cookie and Cracker Technology, AVI Publishing Co, Westport, Conn., Page 176, Table 45 (1968), which table is incorporated herein by reference. The entire food composition can also be lactose-free. The food composition can also contain yogurt culture or be devoid of yogurt culture.

In addition, various components can be added to the composition for flavor and visual appeal, such as, for example, crisped rice, chopped nuts, granola, fruit toppings, dried fruits (e.g., raisins, figs, dates, apricots, banana chips), seeds (e.g., sesame seeds, sunflower seeds), chocolate filling, milk chocolate chips, white chocolate chips, butterscotch chips, peanut butter chips and/or candy chips, etc.

The food composition of this invention can further comprise fillers, starches (e.g., corn starch), softeners, liquefiers, wetting agents, gels (e.g., cellulose gel), other artificial and natural flavorings, glycerin, compound coatings, food dyes, preservatives, carriers, diluents, binders, milk fats, sodium caseinate, titanium dioxide, calcium caseinate, nonfat dry milk, modifiers (e.g., lactose), humectants, hydrophilic film formers, thickening agents, methyl cellulose, carageneenan, carboxymethylcellulose, xantham gum, milk solids, water, lecithin, digestive enzymes (e.g., alpha galactosidase), citric acid, potassium, chloride, calcium phosphate, coloring agents, and/or any combination thereof, as these components are known in the art of food production to enhance food appearance and taste and facilitate packaging and shelf life of the food product at room temperature and/or refrigerator temperature. The food composition of this invention can also be devoid of any of these components or any detectable amount of these components, singly or in any combination. As a nonlimiting example, the food composition can comprise various components as described herein with the proviso that the food composition does not contain any detectable amount of a digestive enzyme.

The term “chocolate” as used herein is intended to refer to all chocolate or chocolate-like compositions with a fat phase or fat-like composition. In the United States, chocolate is subject to a standard of identity (SOI) established by the U.S. Food and Drug Administration under the Federal Food, Drug and Cosmetic Act. Definitions and standards for the various types of chocolate are well established in the U.S. Nonstandardized chocolates are those chocolates that have compositions that fall outside the specified ranges of the standardized chocolates. The term “chocolate,” as used herein, is intended to include standardized and non-standardized chocolates, i.e., including chocolates with compositions conforming to the SOI and compositions not conforming to the SOI, including dark chocolate, baking chocolate, milk chocolate, sweet chocolate, semi-sweet chocolate, buttermilk chocolate, skim-milk chocolate, mixed dairy product chocolate, low fat chocolate, white chocolate, aerated chocolates, compound coatings, non-standardized chocolates and chocolate-like compositions. Chocolate also includes products containing crumb solids or solids fully or partially made by a crumb process. Nonstandardized chocolates result when, for example, the nutritive carbohydrate sweetener is replaced partially or completely; or when the cocoa butter or milk fat are replaced partially or completely; or when components that have flavors that imitate milk, butter or chocolate are added or other additions or deletions in formula are made outside the FDA standards of identity of chocolate or combinations thereof. See, e.g., U.S. Pat. No. 6,521,278.

Chocolate used in the present invention can be a coating chocolate in which the cocoa fat content has been reduced. More particularly, the chocolate can contain no more than 3, 5 or 10 percent by weight of cocoa fat, and at least 10, 20, or 25 percent by weight of vegetable oil (such as partially hydrogenated vegetable oil).

Chocolate with a softening point above 100° F. can be used in the composition of the present invention, and particularly chocolate with a softening point between about 100° F. and about 120° F. The softening point of chocolate can be adjusted by any suitable means, such as by including an alternative source of fat such as partially hydrogenated vegetable oil as opposed to cocoa butter, as some or all of the fat in the chocolate, as noted above.

Humectants that can be included in the food composition of the present invention include, but are not limited to, corn syrup, high fructose corn syrup, polyhydric alcohols (e.g., sorbitol, glycerol, xylitol and the like), polydextrose, and combinations thereof, etc. It will be appreciated that the humectant can also serve a sweetening function.

Flavorants that can optionally be included in the food composition of the present invention include flavors of natural or artificial origin. Example flavors include, but are not limited to, almond nut, amaretto, anisette, brandy, cappuccino, mint, cinnamon, cinnamon almond, crème de menthe, grand mariner, peppermint stick, pistachio, sambuca, apple, chamomile, cinnamon spice, creme, creme de menthe, vanilla, French vanilla, Irish creme, kahlua, lemon, macadamia nut, orange, orange leaf, peach, strawberry, grape, raspberry, cherry, coffee, chocolate and the like, aroma enhancers such as acetaldehyde, herbs, spices, mocha, butternut, rum, hazelnut, horchata, dulche de leche, etc., as well as mixtures of these flavors in any combination See, e.g., U.S. Pat. No. 6,207,206.

The food compositions of this invention, in solid, semi-liquid or liquid form, or as a dietary supplement can be made and packaged according to art-known methods of food manufacture and production. See, e.g., U.S. Pat. No. 6,676,982, U.S. Pat. No. 4,859,475 and U.S. Pat. No. 6, 132, 795, the entire contents of each of which are incorporated by reference herein for disclosures of methods of making food compositions and components of food compositions.

The present invention is more particularly described in the following examples that are intended as illustrative only since numerous modifications and variations therein will be apparent to those skilled in the art.

### EXAMPLES

### Example 1

### Atherosclerosis

Mice and diets. The mice used in these studies were bred and reared in animal facilities, which are fully accredited by the Association for the Assessment and Accreditation of Laboratory Animal Care. All procedures involving animals were approved by the Institutional Animal Care and Use Committee of Wake Forest University School of Medicine. The original breeding pair of LDL receptor −/−, human apoB transgenic mice (9) was provided by Dr. Helen Hobbs, Departments of Internal Medicine and Molecular Genetics, University of Texas Southwestern Medical Center, Dallas, Tex. This mouse is a hybrid cross between the LDL receptor −/− mouse (10) which is itself a hybrid of 129sv and C57BL/6 strains and the human apoB transgenic mouse (11) a hybrid of SJL and C57BL/6B strains. ApoE −/− mice (12), backcrossed >99% to C57BL6/J were provided by Dr. Nobuyo Maeda, Department of Pathology and Laboratory Medicine, University of North Carolina at Chapel Hill, Chapel Hill, N.C.

At six weeks of age, male and ovariectomized female mice of each genotype were assigned randomly to ten diet groups. There were 10 to 16 mice of each sex and genotype in each diet group (total n=416).

The diets are described in Table 1. The principal difference between the diets was the source of the protein component. Casein/lactalbumin was used in an isoflavone-free, soy protein-free control diet. The soy protein sources varied in respect to their isoflavone content. Therefore, to control for the possibility that differences in isoflavone content may contribute to differences in effects of protein source, the protein sources were fed to separate groups of animals 1) in the form they were produced and 2) with isoflavone concentrate added in amounts sufficient to equalize isoflavone intake across soy protein groups. The total isoflavone content of these diets was equivalent to the amount (0.1 mg/Cal) consumed by mice fed the diet with isoflavone-containing soy protein isolate. Isoflavone concentrate was produced by ethanol extraction of defatted soy flake. The β-conglycinin and glycinin protein sources were made from defatted soy flake using the typical isolate process with the exception of precipitation pH. This soy protein isolate was then fractionated based on isoelectric points of the various protein types and the fractions were spray-dried. W008 was produced using the typical isolate process with an added hydrolysis step and the hydrolyzed precipitated fraction was spray-dried for incorporation into the experimental diets. The β-conglycinin-free protein source was made using the typical isolate process with a cultivar of soybean that produces no β-conglycinin. Soy protein isolates and isoflavone concentrates were provided by the Solae Company, St. Louis, Mo. Isoflavone content of these products was determined by high-performance liquid chromatography-mass spectroscopy (13) in the Nestle Purina Analytical Laboratory, St. Louis, Mo.

After 16 weeks, mice were anesthetized with ketamine (80 mg/kg) and xylazine (8 mg/kg) and one mL of blood was collected by cardiac puncture. Mice were then killed with pentobarbital (200 mg/kg). The heart and aorta were promptly removed and placed in 10% neutral buffered formalin for subsequent processing. Plasma was promptly separated at 5000×g and stored at −20° C.

Atherosclerosis and plasma lipoproteins. Plasma lipoproteins were separated by high-performance liquid chromatography (14) and aliquots of isolated lipoprotein fractions were used for enzymatic determination of cholesterol (15).

Analysis for aortic free and esterified cholesterol content was conducted as described previously (16). The aorta was placed on the platform of a dissecting microscope and the adventitia was carefully and completely dissected away from the intima/media and removed. The intima/media was then placed in three mL of chloroform/methanol (2:1, v/v) containing 5 α-cholestane as an internal standard and the lipids were extracted. The lipid extract was separated by filtration and extracts were dried under N₂ at 60° C. and then dissolved in hexane. Analysis of free and total cholesterol was done with two injections per sample on a DB 17 (0.53 mm i.d.×15 m×1 μm) gas-liquid chromatography column at 250° C. and installed in a Hewlett Packard (Palo Alto, Calif.) 5890 gas chromatograph equipped with an HP 7673A automatic injector using on-column injection and a flame ionization detector. Cholesteryl ester was calculated as the difference between free and total cholesterol, as measured before and after saponification and reextraction of the nonsaponifiable sterol into hexane. The delipidated tissue protein was then digested and dissolved in 1 mol/L NaOH and total protein was determined (17).

Data Analysis. To reduce skewness and equalize group variances, all data sets underwent logarithmic transformation before analysis. Three-way (protein type by sex by isoflavone content), two-way (protein type by sex) and one-way (protein type within each sex) analysis of variance were used for detecting effects of protein type, isoflavone content and sex on atherosclerosis and plasma lipoproteins. Pairwise comparisons were made using Duncan's New Multiple Range Test. Multiple linear regression was used to assess the relationship between effects of treatment on plasma lipoproteins and effects on atherosclerosis and to select covariates for analysis of covariance. Analyses were done using BMDP Statistical Software (University of California, Berkeley, Calif.). Results are presented as mean±SEM. Differences were considered significant at P<0.05.

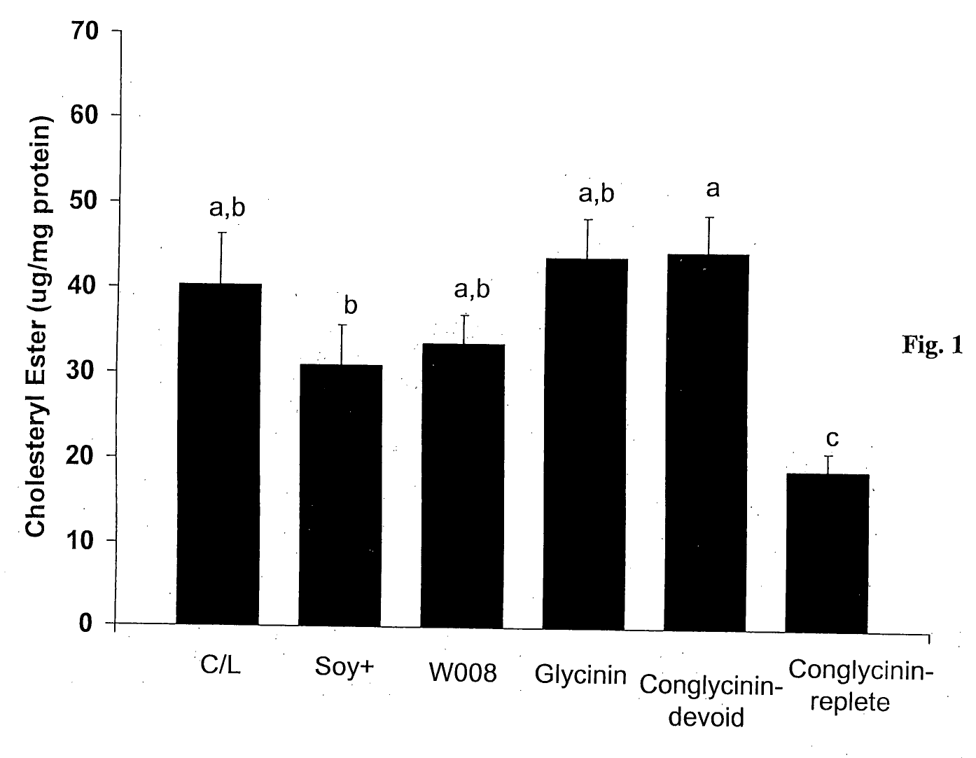

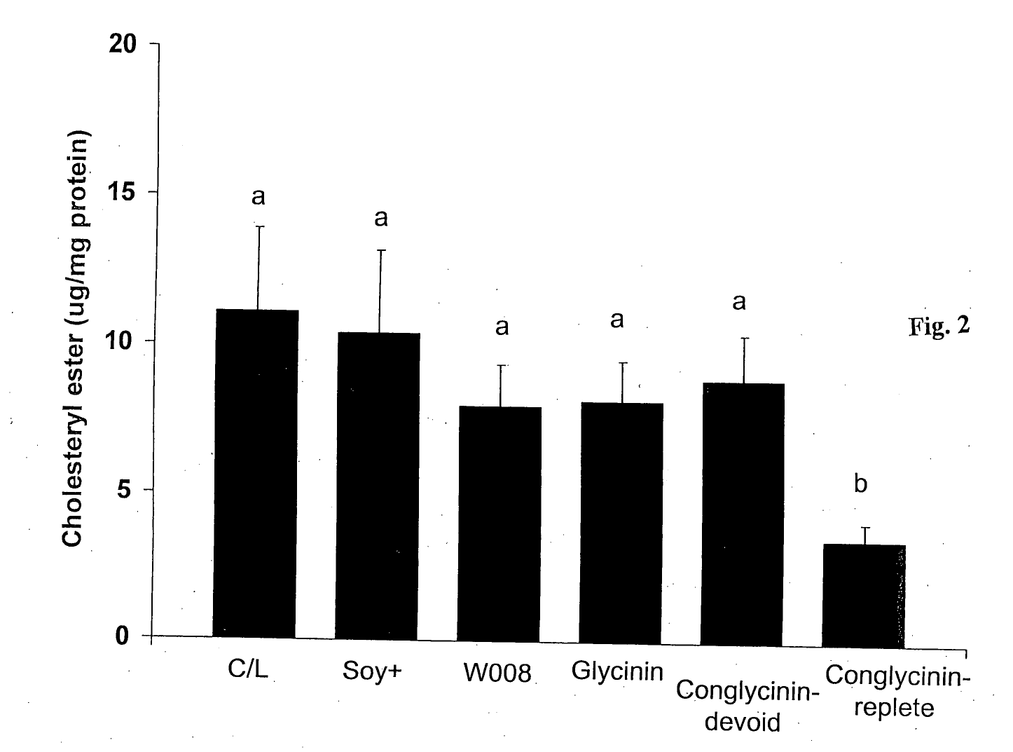

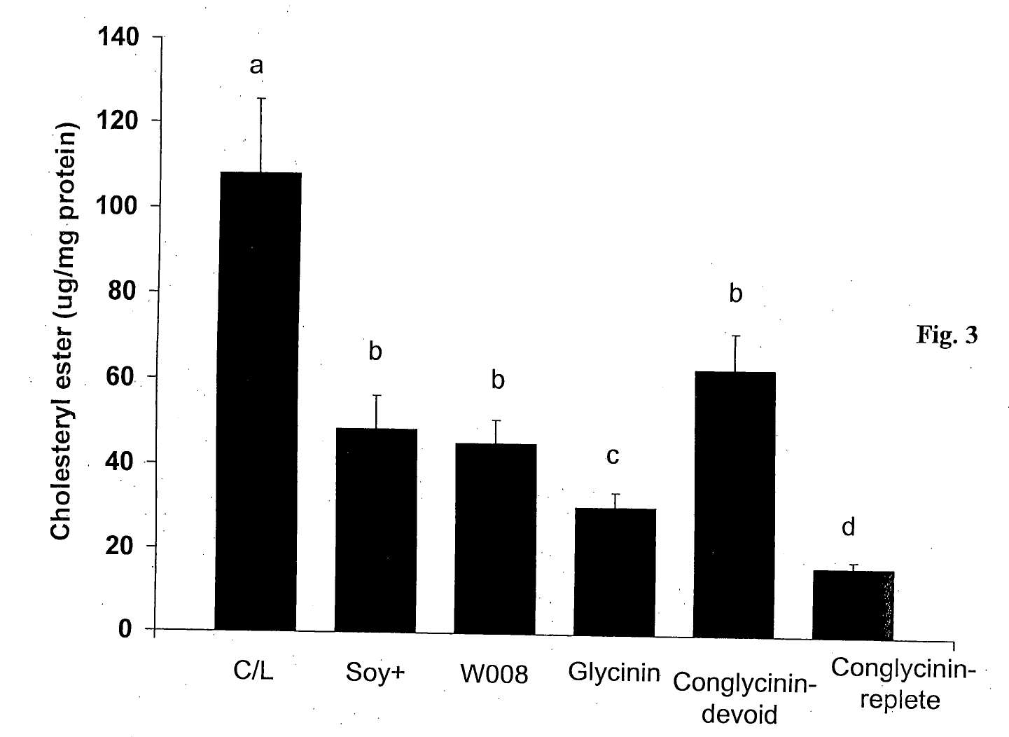

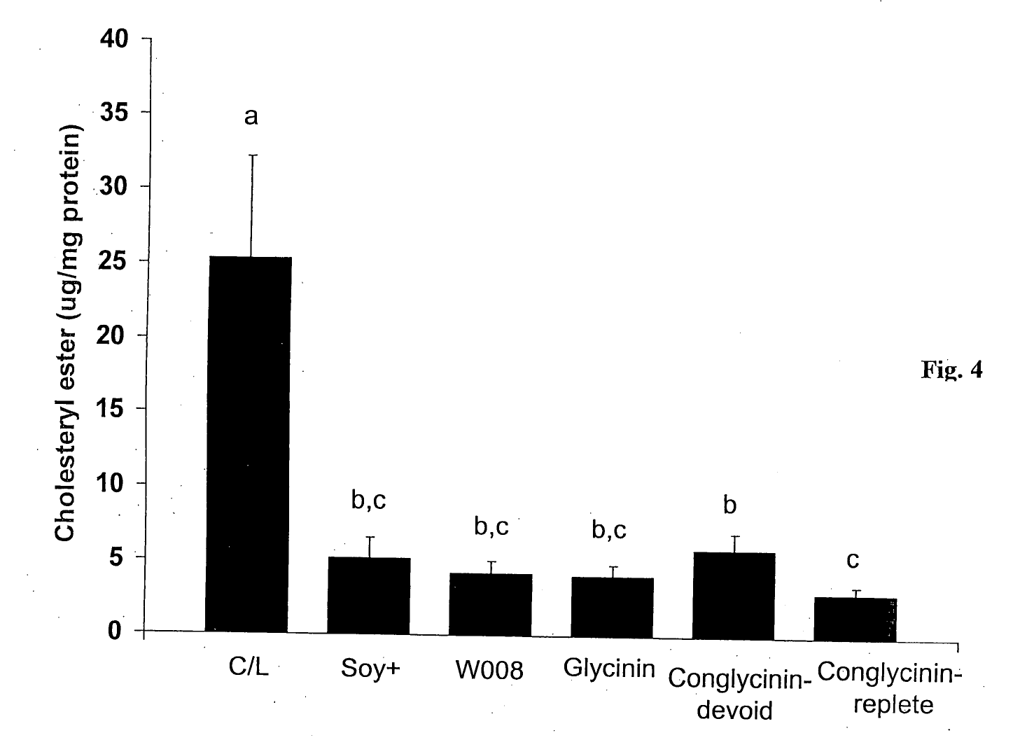

Atherosclerosis extent was four to five times greater in apoE −/− mice than LDL receptor −/−, apoB transgenic mice (FIGS. 1 to 4). For this reason, data were analyzed separately for the two types of mouse. Three-way analysis of variance revealed main effects of protein type (P<0.001) and sex (P<0.02) but not isoflavone content (P>0.2) and a significant sex/protein interaction term (P<0.001) in both types of mouse. As there was no effect of isoflavone content, groups of mice fed diets with and without added isoflavones were combined for further analysis. Patterns of response differed greatly between males and ovariectomized females. Among male mice, atherosclerosis was reduced only in mice fed β-conglycinin (both types of mouse) (P's<0.05) relative to those fed casei lalbumin (FIGS. 1 and 2). Among females, atherosclerosis extent was reduced in both types of mouse in all soy protein groups (P's<0.05) relative to those fed casein/lactalbumin (FIGS. 3 and 4). Relative to mice fed isoflavone-containing soy protein, atherosclerosis extent was reduced only in mice fed β-conglycinin. Among males, atherosclerosis extent was reduced 66% (P<0.05) (LDL receptor −/−) and 39% (P<0.05) (apoE −/−) (FIGS. 1 and 2) while among ovariectomized females, atherosclerosis extent was reduced 67% (P<0.05) in apoE −/− mice (FIGS. 3 and 4).

Plasma lipoproteins. As with atherosclerosis extent, there were main effects of sex, type of mouse and protein type (all P's<0.01) but no effect of isoflavone content (P>0.2). Among female LDL receptor −/− mice, plasma LDL and VLDL+ILDL cholesterol were lower in mice fed all the soy protein-containing diets relative to mice fed the casein/lactalbumin-containing diets while HDL cholesterol was not different (Table 2). Among male LDL receptor −/− mice, while there were no effects on VLDL+ILDL or HDL cholesterol, plasma LDL cholesterol was elevated in the W008 and glycinin groups relative to the casein/lactalbumin group (Table 2). Among female apoE −/− mice, with one exception there were no effects of protein type on any variable. Plasma VLDL+ILDL was elevated in mice in the glycinin group relative to the casein/lactalbumin group (Table 3). Among male apoE −/− mice, plasma LDL cholesterol was reduced in the glycinin group and VLDL+ILDL cholesterol was increased in the W008 group relative to the casein/lactalbumin group. There were no effects of protein type on plasma HDL cholesterol in this group (Table 3).

Multiple linear regression was used to determine which lipoprotein variables were significant predictors of atherosclerosis extent. For LDL receptor −/− mice, LDL cholesterol was a significant predictor accounting for 12% of the variability in atherosclerosis in males and 23% in females. For apoE −/− mice, HDL cholesterol was selected as a significant predictor accounting for 29% of the variability in atherosclerosis extent in males and 14% in females. To determine whether effects of diet on atherosclerosis persist after adjustment for variation in plasma LDL or HDL cholesterol, analysis of covariance with LDL cholesterol (LDL receptor −/− mice) or HDL cholesterol (apoE −/− mice) as a covariate was used. Adjusted means were only trivially different than unadjusted means and main effects of protein type persisted (P's <0.002).

The principle finding was that β-conglycinin, one of the major soy bean storage proteins, had a pronounced inhibitory effect on the development of atherosclerosis that greatly exceeded the effect of isoflavone-containing soy protein isolate. These results represent the first direct in vivo evidence for atheroprotective effects of soy peptides or peptide fractions that have been found previously to have hypolipoproteinemic effects in human beings (3) or experimental animals (1,2,6) and the ability to upregulate LDL receptor activity in vitro (4,5,7). Interestingly, however, neither of these mechanisms seemed to have a substantial role. Since the effects were of similar magnitude in mice with and without LDL receptors, it does not appear that there is a necessary role for LDL receptors. Furthermore, the effects were unrelated or only minimally related to plasma lipoprotein concentrations in both types of mouse. As in previous studies (18-20), plasma total cholesterol and lipoprotein cholesterol concentrations were unaffected by dietary soy protein isolate in apoE −/− mice. In the present study, there was also no effect of dietary β-conglycinin on total or lipoprotein cholesterol concentrations in these mice. Also consistent with a previous study (18), female, but not male, LDL receptor −/−, apoB transgenic mice had reduced total, LDL and VLDL cholesterol concentrations in response to dietary soy protein isolate. In the present study, there was a similar response to β-conglycinin. However, analysis of covariance indicated that atheroprotective effects of soy protein isolate and β-conglycinin were not explained by effects on total, VLDL or LDL cholesterol concentration. Therefore, it seems that the atheroprotective effects of β-conglycinin cannot be accounted for by effects on these atherosclerosis risk factors in either type of mouse.

Isoflavone concentrates were added in relatively small amounts (less than one mg/g of diet) to some diets to equalize isoflavone intake across diet groups. These concentrates were produced by ethanol extraction of defatted soy flakes and, therefore, contained other ethanol-soluble substances that could influence the development of atherosclerosis. Perhaps most notable in this regard are the soyasaponins. Some saponins have hypolipoproteinemic effects (21) and, therefore, have the potential to influence the development of atherosclerosis. The saponin content of this concentrate was not determined. However, the saponin content of a similar concentrate used by Yamakoshi, et al (22) was 11%. In that study (22), there was no effect of the isoflavone concentrate or another saponin-rich concentrate on plasma lipoprotein cholesterol concentrations in rabbits. Furthermore, in the present study there was no difference in atherosclerosis extent between mice fed the diets with isoflavone concentrate and those fed diets without added isoflavone concentrate. Also, the effects of β-conglycinin on atherosclerosis were unexplained by plasma lipoprotein concentrations. Therefore, while saponins may, under some circumstances, influence lipoprotein metabolism and atherosclerosis, it seems unlikely that saponins had an influence on the outcome of this study.

It was surprising to observe that, while isoflavone-containing soy protein isolate had a potent athero-inhibitory effect in female mice, it had no effect in male mice. It is believed that this has to do with the isoflavone content of the isolate used. This isolate contained 0.1 mg of total isoflavones/Cal of diet, an amount approximately 50% higher than used in previous studies (18,19) where atheroinhibitory effects were observed in both male and female mice. There appears to be a range of effective doses above and below which isoflavones are not atheroprotective. This is supported by the findings of Clarkson, et al. (23) who found that among monkeys fed an isoflavone-rich diet there was no inhibition of atherosclerosis in animals with the highest plasma isoflavone concentrations. There may have been no athero-inhibition in male mice because they have plasma isoflavone concentrations two to three times those of females (18). This indicates that plasma isoflavone concentrations were in an optimal range for inhibition of atherosclerosis in the females but were above that range in the males fed this high-isoflavone isolate. It is possible that in the male mice, the plasma isoflavone concentrations achieved at a human dose equivalent of 180 mg/day are beyond a range within which isoflavones inhibit atherosclerosis, i.e., the relationship between plasma isoflavones and atherosclerosis is not linear, but U-shaped, while in the female mice, plasma isoflavone concentrations are effective because they are within the range at the bottom of the U.

Nonetheless, the athero-inhibitory effects of β-conglycinin were pronounced in both males (a mean reduction of 53%) and females (a mean reduction of 56%) relative to mice fed isoflavone-containing soy protein isolate. Among female mice, atherosclerosis extent was reduced an average of 85% relative to female mice fed casein/lactalbumin. The magnitude of the atheroprotective effect of isoflavone-containing β-conglycinin in female mice substantially exceeds that achieved by the feeding of isoflavone-containing soy protein isolate in the current study and a previous study (18). These findings indicate that favorable. cardiovascular effects of soy can be enhanced by the consumption of products rich in β-conglycinin.

In conclusion, the consumption of a diet rich in β-conglycinin has an inhibitory effect on the development of atherosclerosis that greatly exceeds the effect of whole isoflavone-containing soy protein isolate and does not depend on LDL receptors or effects on plasma lipoproteins.

### Example 2

### Osteoporosis

Studies are conducted in which 5 to 30 grams of β conglycinin replaces the usual source of protein in the diet fed daily to ovariectomized adult female cynomolgus monkeys for a period of 24 months. These studies are carried out with and without an effective amount (as described herein) of soy isoflavone included in the diet. A control group receives only the usual source of protein. Animals are fed ad libitum. At the beginning and end of this time period, measurements are made of bone density of the lumbar spine and whole body using dual energy X-ray absorptiometry; and bone density of the femoral neck using peripheral quantitative computerized tomography. At the end of this period, the animals are labeled with calcein for dynamic and static histomorphometric assessments of bone mineral density and bone formation rate on samples of bone obtained at necropsy. An increase or maintenance in bone density in the animals that received β conglycinin, with or without soy isoflavone, as compared to a decrease in bone density in the control animals indicates that the administration of β conglycinin, with or without isoflavone, was effective in treating and/or preventing osteoporosis. The test period can be prolonged and/or repeated and the dose of β conglycinin and/or soy isoflavone can be increased or decreased as described herein.

### Example 3

### Obesity

Studies are conducted in which 5 to 30 grams of β conglycinin replaces the usual source of protein in the diet fed daily to adult female cynomolgus monkeys for a period of 24 months. These studies are carried out with and without an effective amount (as described herein) of soy isoflavone included in the diet. A control group receives only the usual source of protein. Animals are fed ad libitum. At the beginning and end of this time period, measurements are made of body weight and body length; lean body mass and fat mass using dual energy X-ray absorptiometry; and body fat distribution using computerized tomography. A decrease or maintenance in body weight, body length, and/or fat mass in the animals that received β conglycinin, with or without soy isoflavone, as compared to an increase in body weight, body length and/or fat mass in the control animals indicates that the administration of β conglycinin, with or without soy isoflavone, was effective in treating and/or preventing obesity. The test period can be prolonged and/or repeated and the dose of β conglycinin and/or soy isoflavone can be increased or decreased as described herein.

### Example 4

### Diabetes

Studies are conducted in which 5 to 30 grams of β conglycinin replaces the usual source of protein in the diet fed daily to adult female cynomolgus monkeys for a period of 24 months. These studies are carried out with and without an effective amount (as described herein) of soy isoflavone included in the diet. A control group receives only the usual source of protein. Animals are fed ad libitum. At the beginning and end of this time period, measurements are made of fasting plasma insulin, fasting plasma glucose and the plasma insulin and glucose responses to intravenous glucose tolerance tests. Maintenance or normalizing of glucose and insulin levels in the animals that received β conglycinin, with or without soy isoflavone, as compared to changes in insulin and glucose levels indicative of diabetes in the control animals indicates that the administration of β conglycinin, with or without soy isoflavone, was effective in treating and/or preventing diabetes. The test period can be prolonged and/or repeated and the dose of β conglycinin and/or soy isoflavone can be increased or decreased as described herein.

### Example 5

### Blood Pressure

Studies are carried out in which adult human volunteers are assigned to consume 10 to 100 g of β conglycinin, with or without an effective amount of soy isoflavone, per day for one month. Blood pressure is measured by standard methods before and after treatment. A decrease or maintenance of blood pressure in the subjects that received β conglycinin, with or without soy isoflavone, as compared to an increase in, or failure to maintain, blood pressure in the control group indicates that the β conglycinin, with or without soy isoflavone, was effective in lowering or maintaining blood pressure. The test period can be prolonged and/or repeated and the dose of β conglycinin and/or soy isoflavone can be increased or decreased as described herein.

### Example 6

### Memory and Cognitive Function

Studies are conducted in which 5 to 30 grams of β conglycinin replaces the usual source of protein in the diet fed daily to adult female cynomolgus monkeys for a period of 24 months. These studies are carried out with and without an effective amount (as described herein) of soy isoflavone included in the diet. A control group receives only the usual source of protein. Animals are fed ad libitum. At the beginning and end of the study period, the animals are tested for memory and cognitive function using well established protocols for assessing animals' performance in certain computer tasks that address their capabilities in concept formation, spatial memory, visual discrimination, visual memory and long-term memory. An improvement in, or maintenance of, memory of cognitive function in the animals that received the β conglycinin, with or without soy isoflavone, as compared to a decline in, or lack of maintenance of, memory and cognitive function indicates that the β conglycinin, with or without soy isoflavone, was effective in improving and/or maintaining memory and cognitive function. The test period can be prolonged and/or repeated and the dose of β conglycinin and/or soy isoflavone can be increased or decreased as described herein.

### Example 7

### Cancer

Studies are conducted in which 5 to 30 grams of β conglycinin replaces the usual source of protein in the diet fed daily to adult female cynomolgus monkeys for a period of 24 months. These studies are carried out with and without an effective amount (as described herein) of soy isoflavone included in the diet. A control group receives only the usual source of protein. Animals are fed ad libitum. At the end of the study period, mammary glands, uterus and prostate glands are obtained at necropsy for histomorphometric and immunohistochemical assessments of cell proliferation, epithelial hyperplasia, sex hormone receptor density, apoptotic activity and angiogenic activity. Results of these analyses in animals that received β conglycinin, with or without soy isoflavones, indicating no progression to a precancerous or cancerous state, or indicating a reversal of a previously identified cancerous condition, as compared with control animals, indicates that the β conglycinin, with or without soy isoflavone, was effective in treating and/or preventing cancer in the breast, uterus and/or prostate. The test period can be prolonged and/or repeated and the dose of β conglycinin and/or soy isoflavone can be increased or decreased as described herein.

### Example 8

### Anti-atherogenic (Early Atherosclerosis) Effects of Soy Isoflavones Require Estrogen Receptor

Studies were conducted to determine whether soy isoflavones exert atheroprotective effects through estrogen receptor-dependent processes and if so which receptor subtype (α or β)was involved.

A comparison was made of the effects of diets rich in soy protein that was either isoflavone depleted (alcohol-washed) (0.04 mg/g) or isoflavone-replete (1.72 mg/g) in apoE deficient (ee) mice that had been crossed with ER-α and ER-β-deficient mice to produce double-knockout ααee and ββee mice and ER wild-type controls (AAee and BBee). Male and ovariectomized female mice were studied (n=10 to 17 per treatment group; total n=201).

In mice fed the isoflavone-replete diet, atherosclerosis was reduced 20% to 27% (p's<0.05) in ββee, AAee and BBee mice but was unaffected in α ee mice. This inhibitory effect of isoflavone-replete soy protein was unrelated to sex, total plasma cholesterol, VLDL, LDL, and HDL cholesterol, since there was no effect of diet on any variable. These studies indicate that there is a necessary role for ER-α in mediating the effects of dietary soy isoflavones.

### Example 9

### Characterization of Gene Expression in Mouse Tissues by Quantitative Real Time RT-PCR

Studies were conducted to characterize tissue specific effects of dietary consumption of soy components on gene expression in mice. Quantitative real time RT-PCR studies were performed using mouse specific Taqman® primer-probe reagents (Applied Biosystems) to determine mRNA levels for TNFα and MCP1 targets in mouse liver, heart, and aorta, using 18S rRNA as an internal control. TNFα and MCP1 mRNA were found to be expressed in both heart and aorta. In addition, TNFα and MCP1 mRNA levels (as estimated by qRT-PCR) were highly correlated with one another, suggesting that a coordinate regulation of the expression of these markers occurs in different tissues in the same animal. Mouse-specific primer probe sets for beta-actin and glyceraldehyde-3-phosphate dehydrogenase have also been designed and utilized and hypoxanthine guanine phosphoribosyl transferase (HPRT) has been utilized as internal constitutively expressed controls for the qRT-PCR studies. HPRT shows constant expression during development of arterial disease and is relatively comparable between different organs and tissues. In contrast, the use of 18S rRNA alone may be misleading due to its high abundance and greater stability than 28S rRNA and most mRNAs. GAPDH may not be ideal as a number of studies suggest differential regulation of GAPDH in response to various stimuli. Additional mouse-specific primer probe sets to several desired targets that were not commercially available as pre-developed assays, such as estrogen receptors (ERα and ERβ) and targets derived from DNA array studies have also been developed.

Studies were also conducted to examine the possibility that isoflavone consumption might induce alterations in hepatic expression of inflammatory targets. Animals received a diet rich in soy protein that was either isoflavone depleted (alcohol-washed) (0.04 mg/g) or isoflavone-replete (1.72 mg/g) in apoE deficient (ee) mice that had been crossed with Erα- and ERβ-deficient mice to produce double-knockout ααee and ββee mice and ER wild-type controls (AAee and BBee). Gene expression analyses were restricted to the ovariectomized female mice (n=10 to 17 per treatment group; total n=96). Isoflavone-replete diet effects are expressed as percent difference from control in each of the transgenic models. The atheroinhibitory effect of isoflavones was dependent upon the presence of ERα, as the isoflavone-rich diet had no beneficial effect in the ERα KO (ααee). In contrast to the effects observed in the arteries, these data suggest that isoflavone effects on hepatic expression of VCAM-1 might require the presence of both ERα and ERβ, while effects on MCP-1 and TNF-α expression might be ERβ specific.

### Example 10

### Effective Dose of Isoflavones

It was unexpected to find that there appears to be an upper limit to plasma isoflavone concentration (and therefore isoflavone dose) that is effective in inhibiting early atherosclerosis. As it now appears that there may be an optimal dose range for isoflavones, a study has been carried out to determine the lower limit of effective doses. While past studies have used human dose equivalents of 100 to 180 mg per day, this recently completed study addressed dose equivalents of 3, 20 and 50 mg per day and compared effects of aglycone vs. glycoside isoflavones and the extent to which conversion to equol explains the effects of isoflavones. It is now apparent that doses in the range of 20 to 180 mg per day are effective in ovariectomized female mice and doses in the range of 20 to 100 are effective in male mice.

### Example 11

### Effects of Soy Protein on Plasma Oxidation Markers

Ovariectomized monkeys were fed diets devoid of isoflavones with casein and lactalbumin (C/L) as the protein source, or diets containing soy protein containing about 2 mg/g of isoflavones (soy+). After a 6 month treatment, plasma concentrations of myeloperoxidase (MPO; a neutrophil-derived pro-oxidant), isoprostanes (produced in vivo by a free radical-catalyzed mechanism independent of the cyclooxygenase enzyme), and nitrotyrosine (a NO-derived pro-oxidant species) were measured in the two groups.

Results of these studies indicated that soy protein significantly reduced plasma nitrotyrosine concentrations, while only producing non-significant trends in reduced MPO. There were no effects on isoprostanes.

### Example 12

### Effects of Soy on Superoxide Production

Superoxide anions are produced by the endothelium and by the intima-media of arteries exposed to increased oxidative stress. Superoxide anions interact with NO to produce other pro-oxidant species including peroxynitrite. The effects of dietary soy protein on superoxide anion production were examined (via lucigenin assays) in endothelial cell denuded and non-denuded coronary arteries from monkeys fed soy protein or C/L.

Results of these studies indicated that soy inhibits production of superoxide anions in arteries, primarily in the endothelium.

### Example 13

### Atherosclerotic Plaque Stability/Vulnerability

Studies are conducted in which 100 mg to 1 gram of β conglycinin replaces the usual source of protein in the diet fed daily to male and female apoE null mice for four months. These studies are carried out with and without an effective amount (as described herein) of soy isoflavone included in the diet. A control group receives only the usual source of protein. Animals are fed ad libitum.

Assessments of Atherosclerosis Extent and Plaque Vulnerability

These evaluations have three major foci: brachiocephalic (innominate) artery, peripheral arteries (common carotids and carotid bifurcations; iliac and femoral arteries), and aorta. They include assessments of lesion size and extent as well as morphologic characteristics, lipid content and cellular composition.

Mouse Necropsy Procedures: Mice are anesthetized with ketamine (80 mg/kg) and xylazine (8 mg/kg) and blood is collected by cardiac puncture. Plasma is promptly separated at 5000 g and 4° C. and stored at −20° C. The cardiovascular system is flushed with phosphate-buffered saline and perfusion fixed at 100 mm Hg with 10% neutral buffered formalin for five minutes. A separate set of animals is perfused with Ringers solution, the aortae removed, and processed as described below for RNA and protein characterization. The heart and arterial tree is then dissected out intact and examined for the distribution of grossly visible atherosclerotic lesions in the periphery (carotid, renal, iliac and femoral arteries). In some cases, the aorta is removed and stored in formalin for assessment of lesion extent in the aortic root. The brachiocephalic (innominate) artery is examined as described below. In other cases, aortae are placed in RNAlater® reagent and the adventitia carefully and completely dissected away from the intima/media layer. Adventitial tissues are preserved separately in RNAlater® reagent for exploration of the potential role of perivascular cytokine expression in the progression of atherosclerosis and plaque vulnerability measures.

Mouse Aortic Atherosclerosis: In studies of early atherosclerosis, concentration of cholesteryl ester in the aorta has been quantified as an index of atherosclerosis. This measure correlates strongly with both plaque size at the aortic sinus and surface area involvement of the aorta and is a useful measure of atherosclerosis progression.

Mouse Aortic Lipid Content: Analysis for aortic free and esterified cholesterol content is conducted. Formalin-fixed aortae are placed on the platform of a dissecting microscope and the brachiocephalic artery and an area around its origin is removed for processing as described herein. From the remainder of the aorta, adventitia is carefully and completely dissected away from the intima/media and removed. The intima/media is then placed in three ml. of chloroform/ methanol (2:1, v/v) containing 5 α-cholestane as an internal standard and the lipids are extracted. The lipid extract is separated by filtration and extracts dried under N₂ at 60° C. and then dissolved in hexane. Analysis of free and total cholesterol is done with two injections per sample on a DB 17 (0.53 mm i.d.×15 m×1 μm) gas-liquid chromatography column at 250° C. and installed in a Hewlett Packard (Palo Alto, Calif.) 5890 gas chromatograph equipped with an HP 7673A automatic injector using on-column injection and a flame ionization detector. Cholesteryl ester is calculated as the difference between free and total cholesterol, as measured before and after saponification and reextraction of the nonsaponifiable sterol into hexane. The delipidated tissue protein is then digested and dissolved in 1 mol/L NaOH and total protein is determined.

Mouse Brachiocephalic (Innominate) Artery Atherosclerosis: The brachiocephalic artery of apoE null mice predictably develops an advanced plaque by about five months of age. Notably, this plaque develops characteristics of vulnerable plaques over longer periods of time and has a high incidence of rupture and hemorrhage.

The formalin-fixed brachiocephalic artery is prepared, taking care to obtain the origin, which typically has the most advanced lesion. After embedding in paraffin, serial 5 μm sections are cut beginning at the origin, intermittent sections are stained with Verhoeff van Gieson's stain. Adjacent sections are stained for smooth muscle cells and macrophages using immunohistochemical techniques. The cross-sectional area of each atherosclerotic lesion, media and lumen; as well as the length of the internal and external elastic lamina are measured using computer-assisted morphometric techniques. Areas composed of smooth muscle cells and macrophages are quantified and their location in the plaque described. Lesion characteristics are assessed as follows. The presence or absence of an abluminal fibrous cap and whether it is intact is recorded and its thickness quantified. The presence or absence of a lipid-rich necrotic core is recorded and its area quantified. The presence or absence of the following characteristics are tabulated: buried fibrous caps, fibro-fatty nodules, extracellular cholesterol clefts, collagen-rich areas, proteoglycan-rich areas, medial necrosis, medial erosion, chondrocytic metaplasia, fatty streaks adjacent to or superimposed on plaques, plaque rupture and intraplaque hemorrhage.

A greater proportion of smooth muscle cells, thicker fibrous cap, the absence of a necrotic core, less extracellular cholesterol, larger number of collagen rich areas, less intraplaque hemorrhage and fewer ruptured plaques in beta conglycinin treated animals indicates beta conglycinin treatment led to improved plaque stability.

### Example 14

### Protection Against Ischemia-induced Myocardial Infarction (MI)

Studies are conducted in which 100 mg to 1 gram of β conglycinin replaces the usual source of protein in the diet fed daily to male and female C57BL6/J mice for one month. These studies are carried out with and without an effective amount (as described herein) of soy isoflavone included in the diet. A control group receives only the usual source of protein. Animals are fed ad libitum.

After mice are anesthetized and artificially ventilated, a left thoracotomy is performed to expose the heart. MI is produced by permanent ligation of the left coronary artery with a 7-0 silk suture. Sham-operated mice undergo similar surgery without occlusion of the coronary artery. The number of mice surviving MI is recorded and compared among groups. At one month after MI, the hearts are excised and sliced into 2 transverse sections at a level of the papillary muscles. After photographs of the cross-sections are taken, each myocardial slice is weighed. The lengths of the entire epicardial circumferences and portions of infarcted segments at a level of the papillary muscles are measured using a digitizer. Infarct size is calculated and expressed as a percentage of infarcted area of the epicardial circumference. Greater survival rates and smaller infarction sizes in beta conglycinin treated animals indicates that beta conglycinin was effective in limiting adverse effects of myocardial infarction.

### Example 15

### Protection from Atherosclerosis Induced Impairment of Endothelium Dependent Relaxation

Studies are conducted in which 5 to 30 grams of β conglycinin replaces the usual source of protein in the diet fed daily to adult female cynomolgus monkeys for a period of 24 months. These studies are carried out with and without an effective amount (as described herein) of soy isoflavone included in the diet. A control group receives only the usual source of protein. Animals are fed ad libitum.

Quantitative coronary angiography. Near the end of the treatment period, animals are sedated with ketamine hydrochloride (10-15 mg/kg, intramuscularly) and butorphanol (0.025 mg/kg, intramuscularly). A custom-designed 3F (tapered to 1.8F) catheter is inserted into the left femoral artery and advanced into the left main coronary artery under fluoroscopic guidance. To determine endothelium-dependent and—independent coronary artery responses, serial 2-min infusions of 1) 5% dextrose in water, 2) acetylcholine (10⁻⁸, 10⁻⁷, and 10⁻⁶ mol/L, estimated final concentration in the coronary artery), 5% dextrose in water, and 4) nitroglycerin (15 g/min) are performed. Quantitative coronary angiography (QCA Plus, Sanders Data Systems, Palo Alto, Calif.) is also done. Films are analyzed by an operator who was unaware of the subject's treatment group. Increased dilatory responses in monkeys receiving beta conglycinin indicate that beta conglycinin provides some protection against atherosclerosis-related impairment of endothelium dependent vasodilatory function.

### Example 16

### Anti-inflammatory Activity in Osteoarthritis

Studies are conducted in which 100 mg to 1 gram of β conglycinin replaces the usual source of protein in the diet fed daily to one-month old STR/ort male and female mice for five months. These studies are carried out with and without an effective amount (as described herein) of soy isoflavone included in the diet. A control group receives only the usual source of protein. Animals are fed ad libitum. At the end of the five months, mice are killed and necropsied for studies of osteoarthritic changes, e.g., erosion of cartilage, soft tissue calcification, joint instability, varus deformity, and patellar subluxation in the knee joint. Prevention or inhibition of the development of these changes indicates anti-inflammatory activity of 7S for arthritis.

### Example 17

### Anti-inflammatory Activity in Autoimmune Arthritis

Female BALB/c mice at the age of 24-26 weeks are injected intraperitoneally with 100 μg of cartilage PG (measured as protein) emulsified in dimethyldioctadecylammonium bromide (DDA) adjuvant. Booster injections of the same doses of PG with DDA are givers on days 21 and 42. BALB/c mice develop swelling and redness of one or more limbs 7-10 days after the second or third injection with PG in adjuvant. Arthritis is assessed daily, and inflammation is scored from grade 0 to grade 4 for each paw. At 34-36 weeks of age, mice are euthanized.

Limbs are fixed with 10% neutral formalin. After decalcification with 5% formic acid, they are embedded in paraffin, sectioned in 4 μm slices, and stained with hematoxylin and eosin.

The joints of the mice are examined histologically for erosion of the articular bones and cartilage, associated with proliferation of synovial lining cells and infiltration of inflammatory cells into affected tissues. Reduction of in vivo inflammation scores, inhibition of articular erosion, inhibition of synovial cell proliferation, inhibition of inflammatory cell infiltrates indicate anti-inflammatory activity of 7S in autoimmune arthritis.

The foregoing is considered as illustrative only of the principles of the invention. Further, since numerous modifications and changes will readily occur to those skilled in the art, it is not desired to limit the invention to the exact construction and operation shown and described herein. Therefore, accordingly, all suitable modifications and equivalents fall within the scope of the invention.

All publications, patent applications, patents, patent publications and other references cited herein are incorporated by reference in their entireties for the teachings relevant to the sentence and/or paragraph in which the reference is presented.

### REFERENCES

1. Sugano, M., Yamada, Y., Yoshida, K., Hashimoto, Y., Matsuo, T., & Kimoto M. (1988) The hypocholesterolemic action of the undigested fraction of soybean protein in rats. Atherosclerosis 72:115-122.
2. Sugano, M., Goto, S., Yamada, Y., Yoshida, K., Hashimoto Y., Matsuo, T. & Kimoto M. (1990) Cholesterol-lowering activity of various undigested fractions of soybean protein in rats. J. Nutr. 120: 977-985.
3. Wang, M.-F., Yamamoto, S., Chung, H.-M., Chung, S.-Y., Miyatini, S., Mori, M., Okita, T. & Sugano, M. (1995) Antihypercholesteroemic effect of undigested fraction of soybean protein in young female volunteers. J. Nutr. Sci. Vitaminol. 41: 187-195.
4. Lovati, M. R., Manzoni, C., Corsini A., Granata, A., Frattini, R., Fumagalli, R. & Sirtori, C. R. (1992) Low density lipoprotein receptor activity is modulated by soybean globulins in cell culture. J. Nutr. 122: 1971-1978.
5. Lovati, M. R., Manzoni, C., Corsini, A., Granata, A., Fumagalli, R. & Sirtori, C. R. (1996) 7S globulin from soybean is metabolized in human cell cultures by a specific uptake and degradation system. J. Nutr. 126: 2831-2842.
6. Sirtori, C. R., Even, R., & Lovati, M. R. (1993) Soybean protein diet and plasma cholesterol: from therapy to molecular mechanisms. Ann. N.Y. Acad. Sci. 676: 188-201.
7. Lovati, M., Manzoni, C., Gianazza, E., Arnoldi, A., Kurowska, E., Carroll, K. K. & Sirtori. (2000) Soy protein peptides regulate cholesterol homeostasis in Hep G2 cells. J. Nutr. 130:2543-2549.
8. Gianazza, E., Eberini, I., Arnoldi, A., Wait, R. & Sirtori, C. R. (2003) A proteomic investigation of isolated soy proteins with variable effects in experimental and clinical studies. J. Nutr. 133:9-14.
9. Sanan, D. A, Newland, D. L., Tao, R., Marcovina, S., Wang, J., Mooser, V., Hammer R. E. & Hobbs, H. H. (1998) Low density lipoprotein receptor-negative mice expressing human apolipoprotein B-100 develop complex atherosclerotic lesions on a chow diet: no accentuation by apolipoprotein(a). Proc. Natl. Acad. Sci. U. S. A. 95: 4544-4549.
10. Ishibashi S., Brown, M. S., Goldstein, J. L., Gerard, R. D., Hammer, R. E. & Herz, J. (1993) Hypercholesterolemia in low density lipoprotein receptor knockout mice and its reversal by adenovirus-mediated gene delivery. J. Clin. Invest. 92: 883-893.
11. Linton, M. F., Farese, Jr, R. V., Chiesa, G., Grass, D. S., Chin, P. Hammer, R. E., Hobbs, H. H. & Young, S. G. (1993) Transgenic mice expressing high plasma concentrations of human apolipoprotein B 100 and lipoprotein(a). J. Clin. Invest. 92: 3029-3037.
12. Zhang, S. H., Reddick, R. L., Piedrahita, J. A. & Maeda, N. (1992) Spontaneous hypercholesterolemia and arterial lesions in mice lacking apolipoprotein E. Science 258: 468-471.
13. Coward, L., Smith, M., Kirk, M. & Barnes, S. (1998) Chemical modification of isoflavones in soy foods during cooking and processing. Am. J. Clin. Nutr. 68: 1486S-1491S.
14. Carroll, R. M. & Rudel, L. L. (1983) Lipoprotein separation and low density lipoprotein molecular weight determination using high performance gel-filtration chromatography. J. Lipid Res. 24: 200-207.
15. Allain, C. C., Poon, L. S., Chan C. S. G., Richmond, W. & Fu, P. C. (1974) Enzymatic determination of total serum cholesterol. Clin. Chem. 20: 470-475.
16. Rudel, L. L., Kelly, K., Sawyer, J. K., Shah, R. & Wilson, M. D. (1998) Dietary monounsaturated fatty acids promote aortic atherosclerosis in LDL receptor-null, human apoB 100-overexpressing transgenic mice. Arterioscler. Thromb. Vasc. Biol. 18:1818-1827.
17. Lowry, O. J., Rosebrough, N. J., Farr, A. L. & Randall, R. J. (1951) Protein measurement with the Folin phenol reagent. J. Biol. Chem. 191: 265-275.
18. Adams, M. R., Golden, D. L., Anthony, M. S., Register, T. C. & Williams, J. K. (2001) The inhibitory effect of soy protein isolate on atherosclerosis in mice does not require the presence of LDL receptors or alteration of plasma lipoproteins. J. Nutr. 132:43-49.
19. Adams, M. R., Golden, D. L., Register, T. C., Anthony, M. S., Hodgin, J. B., Maeda, N. & Williams, J. K. (2002) The atheroprotective effect of dietary soy isoflavones in apolipoprotein E −/− mice requires the presence of estrogen receptor-α. Arterioscler. Thromb. Vasc. Biol. 22:1859-1864.
20. Ni, W., Tsuda, Y., Sakono, M., & Imaizumi, K. (1998) Dietary soy protein isolate, compared with casein, reduces atherosclerotic lesion area in apolipoprotein E-deficient mice. J. Nutr. 128: 1884-1889.
21. Potter, S. M. (1995) Overview of the proposed mechanisms for the hypocholesterolemic effects of soy. J. Nutr. 125: 606S-611S.
22. Yamakoshi, J., Piskula, M. K., Izumi, T., Tobe, K., Saito, M., Kataoka, S., Obata, A.. & Ikiuchi, M. (2000) Isoflavone aglycone-rich extract without soy protein attenuates atherosclerosis development in cholesterol-fed rabbits. J. Nutr. 130: 1887-1893.

23. Clarkson, T. B., Anthony, M. S., Smith, M., Wilson, L., & Barnes, S. (2002) A paradoxical association between plasma isoflavone concentrations on a soy-containing diet, and both plasma lipoproteins and atherosclerosis. J. Nutr. 132: 583S-584S.

TABLE 1
Compositions (g/kg dry weight) of mouse diets containing different sources of protein

| | | | | | | | Congly- | Congly- | | |
|---|---|---|---|---|---|---|---|---|---|---|
| | C/L | Soy+ | WOO8 | WOO8 +IF ⁷ | Gly | Gly +IF ⁷ | free | free +IF ⁷ | Congly | Congly +IF ⁷ |
| Casein | 105 | — | — | — | — | — | — | — | — | — |
| Lactalbumin | 100 | — | — | — | — | — | — | — | — | — |
| ¹ Soy protein isolate | — | 210.5 | — | — | — | — | — | — | — | — |
| ² WOO8 | — | — | 212 | 212 | — | — | — | — | — | — |
| ³ Glycinin | — | — | — | — | 226 | 226 | — | — | — | — |
| ⁴ β-conglycinin- | — | — | — | — | — | — | 200.3 | 200.3 | — | — |
| devoid soy protein | | | | | | | | | | |
| ⁵ β-conglycinin | — | — | — | — | — | — | — | — | 203.1 | 203.1 |
| DL-Methionine | — | 3 | 3 | 3 | 3 | 3 | 3 | 3 | 3 | 3 |
| Dextrin | 306 | 300.1 | 303.2 | 302.6 | 283.8 | 283.7 | 306 | 305.4 | 299.3 | 299.0 |
| Sucrose | 280 | 280 | 280 | 280 | 280 | 280 | 280 | 280 | 280 | 280 |
| Alphacel | 98.6 | 97 | 101 | 100.7 | 102.5 | 102.5 | 105.7 | 105.4 | 102.4 | 102.2 |
| Lard | 52 | 52 | 52 | 52 | 52 | 52 | 52 | 52 | 53.7 | 53.7 |
| Safflower oil | 10 | 8.9 | .3 | .3 | 4.2 | 4.2 | 4.5 | 4.5 | 10 | 10 |
| Choline bitartrate | 2 | 2 | 2 | 2 | 2 | 2 | 2 | 2 | 2 | 2 |
| Crystalline cholesterol | 1.44 | 1.46 | 1.46 | 1.46 | 1.46 | 1.46 | 1.46 | 1.46 | 1.46 | 1.46 |
| ⁶ Vitamin Mix, AIN-76A | 10 | 10 | 10 | 10 | 10 | 10 | 10 | 10 | 10 | 10 |
| ⁶ Mineral Mix, AIN-76 | 35 | 35 | 35 | 35 | 35 | 35 | 35 | 35 | 35 | 35 |
| ⁷ IF Concentrate | — | — | — | 0.96 | — | 0.11 | — | 0.96 | — | 0.49 |

¹ Soy protein isolate (85% protein) contains 1.03 mg genistein + 0.16 mg daidzein + 0.16 mg glycitein per gram soy protein isolate (in aglycone units).
² Soy protein hydrolysate (84% protein) WOO8 contains 0.38 mg genistein + 0.18 mg daidzein + 0.06 mg glycitein per gram soy protein isolate (in aglycone units).
³ Glycinin-rich soy protein (79% protein) contains 0.91 mg genistein + 0.60 mg daidzein + 0.14 mg glycitein per gram soy protein isolate (in aglycone units).
⁴ β-conglycinin-devoid soy protein isolate (89% protein) contains 0.41 mg genistein + 0.19 mg daidzein + 0.08 mg glycitein per gram soy protein isolate (in aglycone units).
⁵ β-conglycinin-rich soy protein (88% protein) contains 0.74 mg genistin + 0.46 mg daidzin + 0.12 mg glycitin per gram soy protein isolate (in aglycone units).
⁶ Vitamin Mixture AIN-76A (26, 27) and Mineral Mixture AIN-76 (27) were obtained from Harlan Teklad, Madison, WI.
⁷ IF = isoflavone; Soy Isoflavone Concentrate contains 145.2 mg genistein + 116.3 mg daidzein + 21.7 mg glycitein per gram concentrate (in aglycone units).

TABLE 2
Plasma lipoproteins in LDL receptor −/− mice fed diets with different sources of protein¹

| | Total Plasma | | VLDL + ILDL | | | | | |
|---|---|---|---|---|---|---|---|---|
| | Cholesterol | | Cholesterol | | LDL Cholesterol | | HDL Cholesterol | |
| | Male | Female | Male | Female | Male | Female | Male | Female |
| | mmol/L | | | | | | | |
| Casein/lactalbumin | 22.4 ± 1.8 | 34.2 ± 2.6 a | 5.4 ± 0.8 a, b | 6.1 ± 1.0 a | 15.4 ± 1.4 b | 25.7 ± 1.9 a | 1.6 ± 0.1 | 1.7 ± 0.3 |
| Soy + isoflavones | 23.5 ± 1.0 | 24.5 ± 0.9 b | 3.5 ± 0.3 b | 2.8 ± 0.3 b | 17.9 ± 0.7 a, b | 19.5 ± 0.6 b | 2.1 ± 0.2 | 2.1 ± 0.2 |
| W008 | 26.4 ± 0.8 | 22.7 ± 0.9 b | 4.0 ± 0.2 b | 2.9 ± 0.2 b | 20.4 ± 0.6 a | 17.9 ± 0.8 b, c | 1.9 ± 0.2 | 2.0 ± 0.1 |
| Glycinin | 27.9 ± 1.1 | 21.6 ± 0.8 b | 6.1 ± 0.4 a | 3.1 ± 0.2 b | 20.0 ± 0.8 a | 16.6 ± 0.6 b, c | 1.8 ± 0.1 | 2.0 ± 0.1 |
| β-conglycinin-devoid | 27.0 ± 1.3 | 24.4 ± 1.0 b | 5.5 ± 0.5 a, b | 3.7 ± 0.3 b | 19.5 ± 0.9 a, b | 18.6 ± 0.8 b, c | 2.0 ± 0.1 | 2.1 ± 0.1 |
| soy protein | | | | | | | | |
| β-conglycinin | 24.2 ± 1.2 | 20.3 ± 0.6 b | 5.1 ± 0.4 a, b | 3.0 ± 0.2 b | 20.1 ± 0.8 a, b | 15.3 ± 0.5 c | 1.8 ± 0.1 | 1.9 ± 0.1 |

¹Values are mean ± SEM, n = 10 to 24
Within columns, values labeled with different letters differ (P < 0.05) and a > b > c

TABLE 3
Plasma lipoproteins in ApoE −/− mice fed diets with different sources of protein¹

| | Total Plasma | | VLDL + ILDL | | | | | |
|---|---|---|---|---|---|---|---|---|
| | Cholesterol | | Cholesterol | | LDL Cholesterol | | HDL Cholesterol | |
| | Male | Female | Male | Female | Male | Female | Male | Female |
| | mmol/L | | | | | | | |
| Casein/ | 21.8 ± 1.5 a, b, c | 21.3 ± 1.8 b, c | 13.4 ± 1.0 b | 13.5 ± 1.1 b | 7.4 ± 0.5 a | 6.7 ± 0.7 a, b | 1.0 ± 0.1 a, b | 1.1 ± 0.1 |
| lactalbumin | | | | | | | | |
| Soy + | 25.6 ± 1.6 a, b | 17.5 ± 1.0 c | 17.6 ± 1.4 a, b | 11.0 ± 0.7 b | 7.1 ± 0.4 a, b | 5.6 ± 0.5 b | 1.0 ± 0.1 a, b | 0.8 ± 0.1 |
| isoflavones | | | | | | | | |
| W008 | 27.7 ± 1.4 a | 27.2 ± 1.5 a, b | 18.8 ± 1.2 a | 18.9 ± 1.3 a, b | 7.9 ± 0.3 a | 7.3 ± 0.4 a, b | 1.1 ± 0.1 a, b | 1.0 ± 0.1 |
| Glycinin | 18.3 ± 1.4 b, c | 30.6 ± 1.3 a | 11.7 ± 1.1 b | 21.5 ± 1.0 a | 5.5 ± 0.3 b | 8.0 ± 0.4 a | 1.0 ± 0.1 a, b | 1.1 ± 0.1 |
| β-conglycinin | 21.7 ± 0.7 b | 21.3 ± 1.5 b, c | 14.2 ± 0.6 b | 14.1 ± 1.3 b | 6.5 ± 0.3 a, b | 6.4 ± 0.4 a, b | 1.0 ± 0.1 b | 0.9 ± 0.1 |
| devoid soy | | | | | | | | |
| protein | | | | | | | | |
| β-conglycinin | 18.6 ± 1.5 b, c | 23.7 ± 1.5 b | 11.3 ± 1.1 b | 15.3 ± 1.3 b | 6.0 ± 0.4 a, b | 7.1 ± 0.3 a, b | 1.3 ± 0.1 a | 1.2 ± 0.1 |

¹Values are mean ± SEM, n = 11 to 24
Within columns, values labeled with different letters differ (P < 0.05) and a > b > c

What is claimed is:

1. A method of preventing and/or treating a cardiovascular disorder selected from the group consisting of atherosclerosis, coronary heart disease, myocardial infarction, and stroke in a subject in need thereof, comprising administering to the subject an effective amount of β-conglycinin.

2. The method of claim 1, wherein the subject is a mammal.

3. The method of claim 2, wherein the subject is a human.

4. The method of claim 1, wherein the β-conglycinin is administered to the subject in an amount in the range of about 5 grams to about 100 grams at least once daily.

5. The method of claim 1, wherein the β-conglycinin is administered to the subject over a period of at least six months.

6. The method of claim 1, wherein soy isoflavone is also administered to the subject, before, after and/or simultaneously with the administration of the β-conglycinin.

7. The method of claim 6, wherein the isoflavone is administered to the subject in an amount in the range of about 5 to 200 milligrams at least once daily.
